(19) United States
(12) Patent Application Publication
(10) Pub. No.: US 2021/0268237 A1
(43) Pub. Date: Sep. 2, 2021

(54) Introducer Tool And Methods Thereof

(71) Applicant: C. R. Bard, Inc., Franklin Lakes, NJ (US)

(72) Inventors: Christian Andersen, Kaysville, UT (US); John G. Evans, South Jordan, UT (US)

(21) Appl. No.: 16/973,043

(22) PCT Filed: Jun. 19, 2019
(86) PCT No.: PCT/US2019/038045

§ 371 Date: Dec. 8, 2020

Related U.S. Application Data
(60) Provisional application No. 62/687,440, filed on Jun. 20, 2018.

(51) Int. Cl. A61M 25/06; A61M 25/09

(52) U.S. Cl. CPC A61M 25/0606; A61M 25/0668; A61M 25/09041

(57) ABSTRACT

An introducer tool including a housing, a sheath detachably coupled to the housing, and a needle fixed to the housing. The housing can include a guidewire actuating mechanism configured to extend and retract a guidewire fixed to the guidewire actuating mechanism. The sheath can include a handle, and can be detachably coupled to the housing in a first configuration of the introducer tool designed for venous introduction of the sheath. An end portion of the needle can extend past an end portion of the sheath in the first configuration of the introducer tool. Methods for venous access including obtaining and using the introducer tool are also disclosed.

### PRIORITY

This application claims the benefit of priority to U.S. Provisional Application No. 62/687,440, filed Jun. 20, 2018, which is incorporated by reference in its entirety into this application.

### BACKGROUND

An introducer tool is needed that safely and efficiently introduces a sheath for venous access by one or more additional medical devices including, for example, ports or catheters. Disclosed herein is an introducer tool and methods thereof that address at least the foregoing.

### SUMMARY

Disclosed herein is an introducer tool including, in some embodiments, a housing, a sheath detachably coupled to the housing, and a needle fixed to the housing. The housing includes a guidewire actuating mechanism configured to extend and retract a guidewire fixed to the guidewire actuating mechanism. The sheath includes a handle. The sheath is detachably coupled to the housing in a first configuration of the introducer tool configured for venous introduction of the sheath. An end portion of the needle extends past an end portion of the sheath in the first configuration of the introducer tool.

In some embodiments, the introducer tool also includes a needle-safety mechanism including a needle-safety housing. The needle-safety housing is configured to enclose the end portion of the needle in a second configuration of the introducer tool subsequent to the venous introduction of the sheath. A transition from the first configuration to the second configuration of the introducer tool includes withdrawal of the needle from the sheath.

In some embodiments, the sheath is a pullable sheath configured to be pulled for removal of the sheath subsequent to the venous introduction of the sheath.

In some embodiments, the sheath is a peelable sheath configured to peel apart from the handle to the end portion of the sheath.

In some embodiments, the sheath has a pair of longitudinal seams or weakened areas enabling the sheath to peel apart from the handle to the end portion of the sheath.

In some embodiments, the sheath includes a polymer in which polymer chains of the polymer are longitudinally oriented enabling the sheath to peel apart from the handle to the end portion of the sheath.

In some embodiments, the end portion of the needle includes a beveled section and a dilator section. The beveled section of the needle is configured to puncture tissue. The dilator section of the needle is configured to dilate the tissue after it is punctured.

In some embodiments, the introducer tool also includes a dilator configured to dilate the tissue after it is punctured with the needle.

Also disclosed herein is a method for venous access including, in some embodiments, obtaining a venous introducer tool. The introducer tool includes, a housing, a sheath detachably coupled to the housing, and a needle fixed to the housing. The housing includes a guidewire actuating mechanism configured to extend and retract a guidewire fixed to the guidewire actuating mechanism. The sheath includes a handle. The sheath is detachably coupled to the housing in an initial configuration of the introducer tool. An end portion of the needle extends past an end portion of the sheath in the initial configuration of the introducer tool. The method also includes puncturing a vein of a patient with the needle of the introducer tool to gain venous access by way of a puncture; extending the guidewire into the vein past the end portion of the needle with the guidewire actuating mechanism; and introducing the sheath into the vein by sliding the sheath over the guidewire.

In some embodiments, the method also includes dilating the puncture with a dilator section of the needle. The needle includes a beveled section for the puncturing and a dilator section for the dilating.

In some embodiments, the method also includes activating a needle-safety mechanism of the introducer tool. Activating the needle-safety mechanism allows for simultaneously withdrawing the needle from the puncture and enclosing at least the beveled section of the needle in a needle-safety housing of the needle-safety mechanism.

In some embodiments, the method also includes withdrawing the guidewire while leaving the sheath in place.

In some embodiments, the method also includes inserting one or more medical devices into the vein of the patient through the sheath and removing the sheath from the vein.

In some embodiments, the one or more medical devices include at least a catheter. Removing the sheath includes withdrawing the sheath from the vein while the catheter is disposed within the sheath and peeling the sheath apart from the handle to the end portion of the sheath.

Also disclosed herein is a method for venous access including, in some embodiments, obtaining an introducer tool. The introducer tool includes, a housing, a peelable sheath detachably coupled to the housing, and a needle fixed to the housing. The housing includes a guidewire actuating mechanism configured to extend and retract a guidewire fixed to the guidewire actuating mechanism. The sheath is detachably coupled to the housing in an initial configuration of the introducer tool. The sheath includes a handle, and the sheath is configured to peel apart from the handle to an end portion of the sheath. An end portion of the needle includes a beveled section and a dilator section. The method also includes puncturing a vein of a patient with the beveled section of the needle of the introducer tool to gain venous access by way of a puncture. The end portion of the needle extends past the end portion of the sheath of the introducer tool for the puncturing of the vein. The method also includes dilating the puncture with the dilator section of the needle; extending the guidewire into the vein past the end portion of the needle with the guidewire actuating mechanism; introducing the sheath into the vein by sliding the sheath over the guidewire; activating a needle-safety mechanism of the introducer tool; simultaneously withdrawing the needle from the puncture and enclosing at least the beveled section of the needle in a needle-safety housing of the needle-safety mechanism; withdrawing the guidewire while leaving the sheath in place; inserting a catheter into the vein through the sheath; and removing the sheath from the vein. Removing the sheath includes withdrawing the sheath from the vein while the catheter is disposed within the sheath and peeling the sheath apart from the handle to the end portion of the sheath.

In some embodiments, the sheath has a pair of longitudinal seams or weakened areas enabling the sheath to peel apart from the handle to the end portion of the sheath.

In some embodiments, the sheath includes a polymer in which polymer chains of the polymer are longitudinally oriented along the sheath enabling the sheath to peel apart from the handle to the end portion of the sheath.

These and other features of the concepts provided herein will become more apparent to those of skill in the art in view of the accompanying drawings and following description, which disclose particular embodiments of such concepts in greater detail.

### DRAWINGS

FIG. 1 illustrates an introducer tool in a first configuration in accordance with some embodiments.

FIG. 2 illustrates the introducer tool in a second configuration in accordance with some embodiments.

FIG. 3 illustrates the introducer tool in a third configuration accordance with some embodiments.

FIG. 4 illustrates the introducer tool in a fourth configuration accordance with some embodiments.

FIG. 5 illustrates an end portion of a needle of the introducer tool in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

Again, an introducer tool is needed that safely and efficiently introduces a sheath for venous access by one or more additional medical devices including, for example, ports or catheters. Disclosed herein is an introducer tool and methods thereof that address at least the foregoing.

### Introducer Tool

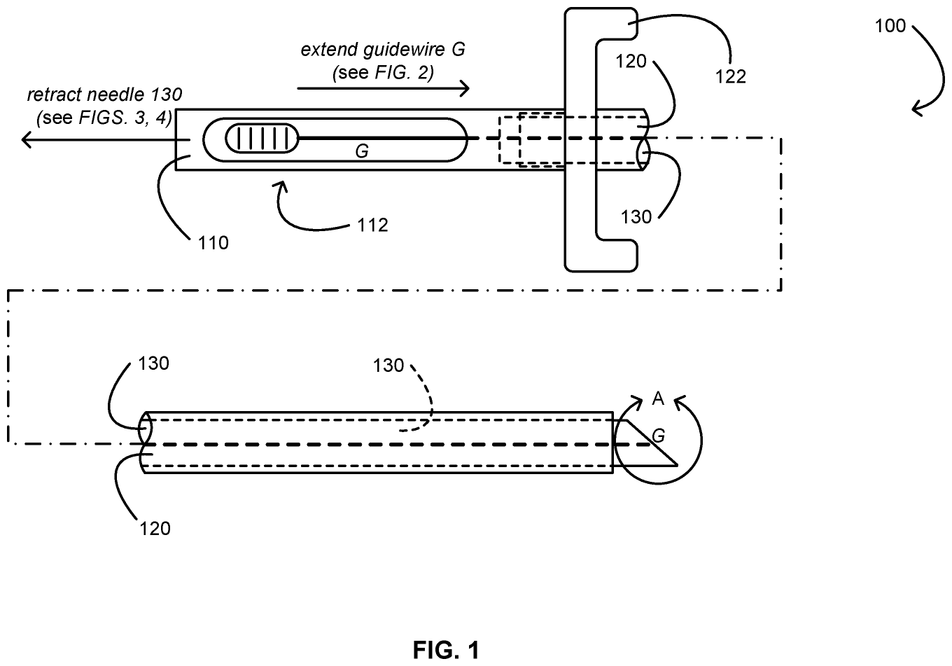

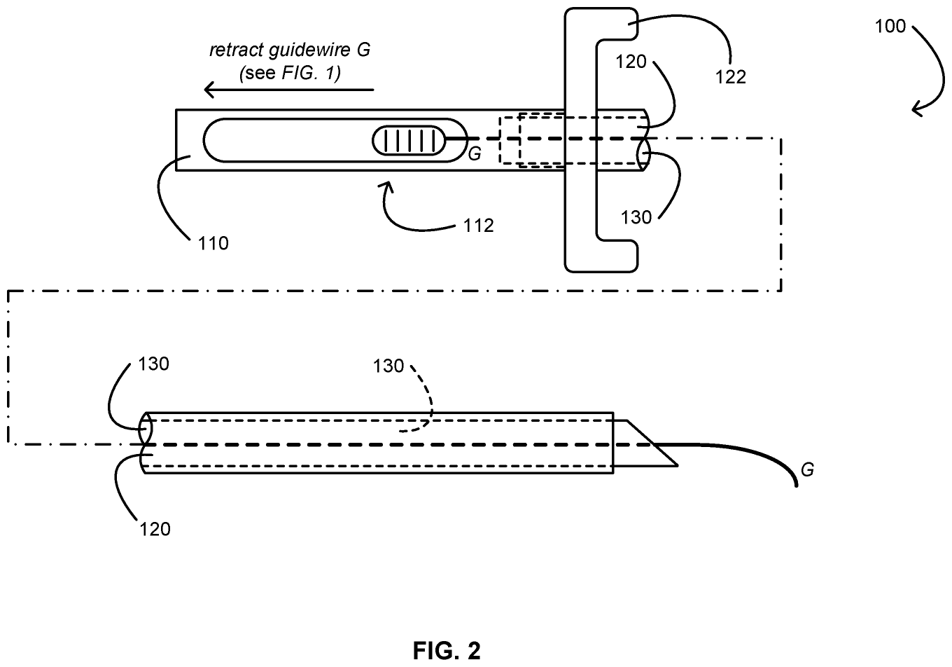

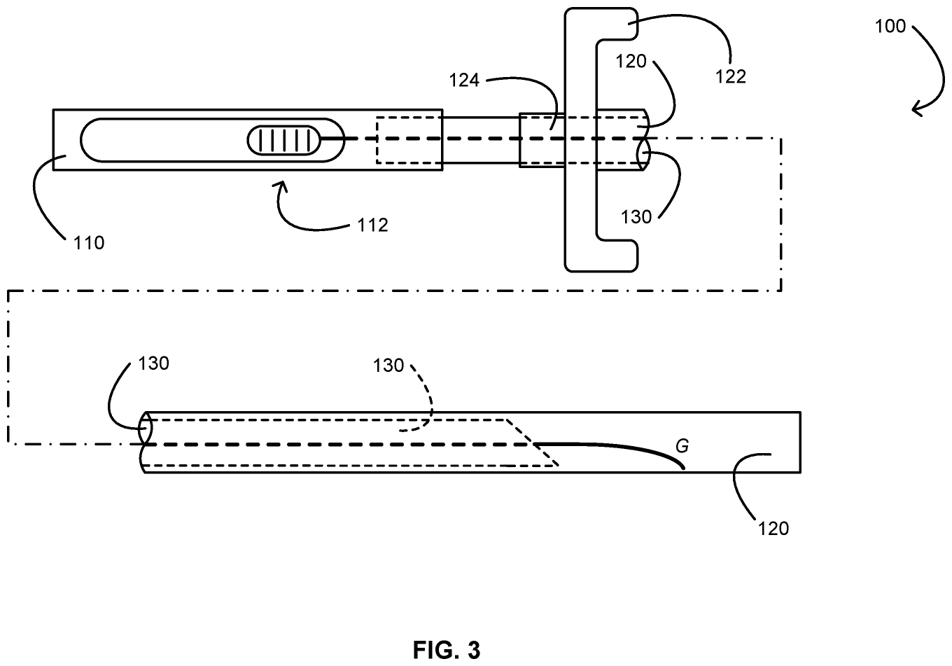

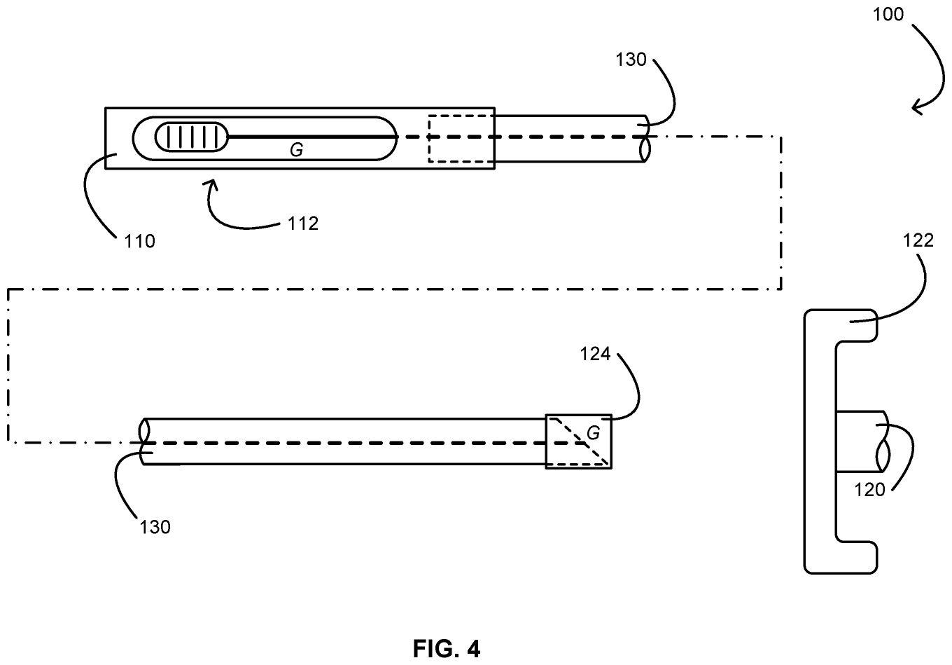

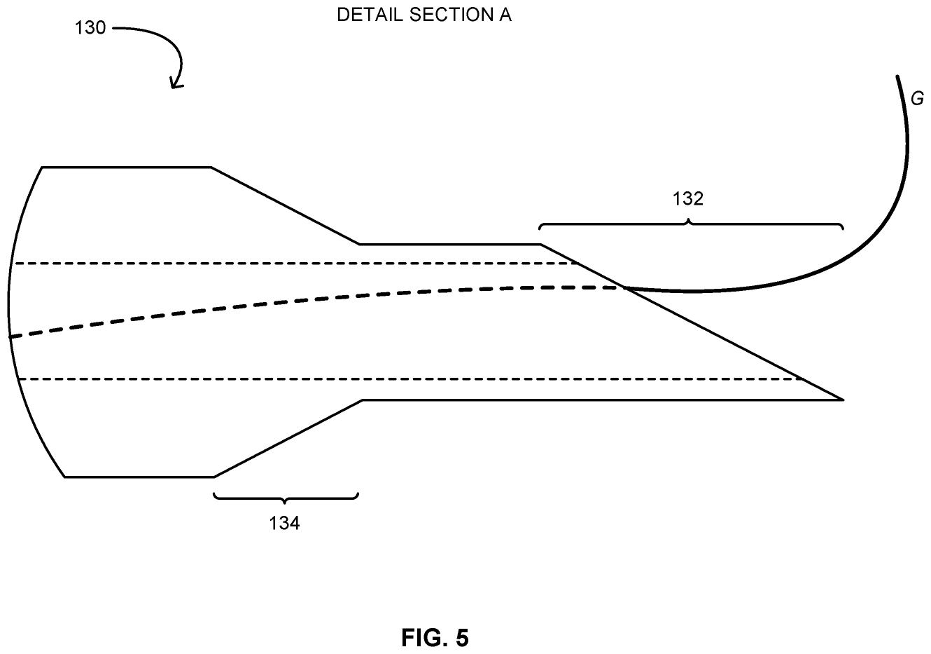

FIGS. 1-4 illustrate an introducer tool 100 in a number of different configurations in accordance with some embodiments. FIG. 1 illustrates the introducer tool 100 in a first or initial configuration in which a housing 110 is coupled to a sheath 120, a guidewire G is completely retracted inside a needle 130, and an end portion of the needle 130 extends from the sheath 120. FIG. 2 illustrates the introducer tool 100 in a second configuration in which the housing 110 is coupled to the sheath 120, an end portion of the guidewire G is extended beyond the end portion of the needle 130, and the end portion of the needle 130 extends from the sheath 120. FIG. 3 illustrates the introducer tool 100 in a third configuration in which the sheath 120 is advanced over the guidewire G, the needle 130 and the guidewire G are partially retracted inside the sheath 120, and the housing 110 is uncoupled from the sheath 120. FIG. 4 illustrates the introducer tool 100 in a fourth configuration in which the housing 110 is completely uncoupled from the sheath 120 and the end portion of the needle 130 is enclosed in a needle-safety housing 124. FIG. 5 illustrates the end portion of the needle 130 of the introducer in accordance with some embodiments.

It should be understood that FIGS. 1-5 illustrate the introducer tool 100 in a simplified matter. Some of the features of the introducer will become more apparent to those of skill in the art in view of one or more references selected from U.S. Pat. Nos. 9,872,971, 8,932,258, 8,998,852, 9,950,139, US 2015/0119806, US 2016/0256667, and US 2018/0028780, each of which is hereby incorporated by reference in its entirety into this application.

As shown in FIGS. 1-4, the introducer tool 100 can include a housing 110, a sheath 120 detachably coupled to the housing 110, and a needle 130 fixed to the housing 110.

The housing 110 can include a guidewire actuating mechanism 112 configured to extend and retract a guidewire G fixed to the guidewire actuating mechanism 112. As shown, the guidewire actuating mechanism 112 can include a slideable button or the like. The guidewire actuating mechanism 112 can be similar to the guidewire advancement assembly of the catheter placement device of at least US 2018/0028780 of the foregoing references.

The sheath 120 can be detachably coupled to the housing 110 in at least the first configuration of the introducer tool 100, which configuration is configured for venous introduction of the sheath 120. The sheath 120 can include a handle 122. The sheath 120 can be a peelable sheath configured to peel apart from the handle 122 to the end portion of the sheath 120. The sheath 120 can have a pair of longitudinal seams or weakened areas enabling the sheath 120 to peel apart from the handle 122 to the end portion of the sheath 120. The sheath 120 can include a polymer (e.g., expanded polytetrafluoroethylene [“ePTFE”]) in which polymer chains of the polymer are longitudinally oriented enabling the sheath 120 to peel apart from the handle 122 to the end portion of the sheath 120. The sheath 120 can include the longitudinal seams or weakened areas, the longitudinally oriented polymer chains, or both. That said, the sheath 120 need not be a peelable sheath. Instead, the sheath 120 can be a pullable sheath configured for pulling the sheath as a method for removing the sheath 120.

An end portion of the needle 130 can extend past an end portion of the sheath 120 in the first configuration of the introducer tool 100. As shown in FIG. 5, the end portion of the needle 130 can include a beveled section 132 and a dilator section 134. The beveled section 132 of the needle 130 is configured to puncture tissue. The dilator section 134 of the needle 130 is configured to dilate the tissue after it is punctured. To allow introduction of a sheath (e.g., the sheath 120) having a diameter large enough to accommodate an 8-Fr catheter, the needle 130 can be manufactured to dilate from a diameter smaller than 8 Fr proximal to the beveled section to a diameter greater than 8 Fr proximal to the dilator section 134. However, the needle 130 need not include the dilator section 134. In such embodiments, a separate dilator can be used to accommodate up to at least the 8-Fr catheter.

The introducer tool 100 can include a needle-safety mechanism including a needle-safety housing 124. The needle-safety housing 124 can be configured to enclose the end portion of the needle 130 in another configuration (e.g., the fourth configuration) of the introducer tool 100 subsequent to the venous introduction of the sheath 120. The needle-safety housing 124 of the needle-safety mechanism can be similar to the safety housing of the catheter placement device of at least US 2018/0028780 of the foregoing references.

A transition from the first configuration of the introducer tool 100 (see FIG. 1) to the second configuration of the introducer tool 100 (see FIG. 2) includes extension of the guidewire G. A transition from the second configuration of the introducer tool 100 (see FIG. 2) to the third configuration of the introducer tool 100 (see FIG. 3) includes advancement of the sheath 120 over the needle 130 and the guidewire G. A transition from the third configuration of the introducer tool 100 (see FIG. 3) to the fourth configuration of the introducer tool 100 (see FIG. 4) includes withdrawal of the needle 130 from the sheath 120.

### Methods

Also disclosed herein is a method for venous access including, in some embodiments, obtaining the introducer tool 100; puncturing a vein of a patient with the needle 130 of the introducer tool 100 to gain venous access by way of a puncture; extending the guidewire G into the vein past the end portion of the needle 130 with the guidewire actuating mechanism 112; and introducing the sheath 120 into the vein by sliding the sheath 120 over the guidewire G.

After puncturing the vein of the patient, the puncture, or the tissue thereof, can be dilate using the dilator section 134 of the needle 130.

After sliding the sheath 120 over the guidewire G, the needle-safety mechanism of the introducer tool 100 can be activated. Activating the needle-safety mechanism allows for simultaneously withdrawing the needle 130 from the puncture and enclosing at least the beveled section 132 of the needle 130 in the needle-safety housing 124 of the needle-safety mechanism.

At a time of or after withdrawing the needle 130 from the puncture, the guidewire G can be withdrawn, thereby leaving the sheath 120 in place.

With the sheath 120 in place, one or more medical devices can be inserted into the vein of the patient through the sheath 120. The one or more medical devices can include at least a port or a catheter. After inserting the one or more medical devices into the vein of the patient, the sheath 120 can be removed. Removing the sheath 120 includes withdrawing the sheath 120 from the vein while the port is disposed beyond the end portion of the sheath 120 or the catheter is disposed within the sheath 120. Removing the sheath includes either withdrawing the sheath 120 or peeling the sheath 120 apart from the handle 122 to the end portion of the sheath 120.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An introducer tool, comprising:
a housing including a guidewire actuating mechanism configured to extend and retract a guidewire fixed to the guidewire actuating mechanism;
a sheath including a handle,
wherein the sheath is detachably coupled to the housing in a first configuration of the introducer tool configured for venous introduction of the sheath; and
a needle fixed to the housing,
wherein an end portion of the needle extends past an end portion of the sheath in the first configuration of the introducer tool.

2. The introducer tool of claim 1, further comprising a needle-safety mechanism including a needle-safety housing configured to enclose the end portion of the needle in a second configuration of the introducer tool subsequent to the venous introduction of the sheath, wherein a transition from the first configuration to the second configuration of the introducer tool includes withdrawal of the needle from the sheath.

3. The introducer tool of claim 1, wherein the sheath is a pullable sheath configured to be pulled for removal of the sheath subsequent to the venous introduction of the sheath.

4. The introducer tool of claim 1, wherein the sheath is a peelable sheath configured to peel apart from the handle to the end portion of the sheath.

5. The introducer tool of claim 4, wherein the sheath has a pair of longitudinal seams or weakened areas enabling the sheath to peel apart from the handle to the end portion of the sheath.

6. The introducer tool of claim 4, wherein the sheath includes a polymer in which polymer chains of the polymer are longitudinally oriented enabling the sheath to peel apart from the handle to the end portion of the sheath.

7. The introducer tool of claim 1, wherein the end portion of the needle includes a beveled section and a dilator section, wherein the beveled section of the needle is configured to puncture tissue, and wherein the dilator section of the needle is configured to dilate the tissue after it is punctured.

8. The introducer tool of claim 1, further comprising a dilator configured to dilate the tissue after it is punctured with the needle.

9. A method for venous access, comprising:
obtaining a venous introducer tool including
a housing including a guidewire actuating mechanism configured to extend and retract a guidewire fixed to the guidewire actuating mechanism;
a sheath including a handle,
wherein the sheath is detachably coupled to the housing in an initial configuration of the introducer tool; and
a needle fixed to the housing,
wherein an end portion of the needle extends past an end portion of the sheath in the initial configuration of the introducer tool;
puncturing a vein of a patient with the needle of the introducer tool to gain venous access by way of a puncture;
extending the guidewire into the vein past the end portion of the needle with the guidewire actuating mechanism; and
introducing the sheath into the vein by sliding the sheath over the guidewire.

10. The method of claim 9, further comprising dilating the puncture with a dilator section of the needle, wherein the needle includes a beveled section for the puncturing and a dilator section for the dilating.

11. The method of claim 9, further comprising activating a needle-safety mechanism of the introducer tool; and simultaneously withdrawing the needle from the puncture and enclosing at least the beveled section of the needle in a needle-safety housing of the needle-safety mechanism.

12. The method of claim 9, further comprising withdrawing the guidewire while leaving the sheath in place.

13. The method of claim 12, further comprising inserting one or more medical devices into the vein of the patient through the sheath; and removing the sheath from the vein.

14. The method of claim 13, wherein the one or more medical devices include at least a catheter, and wherein removing the sheath includes withdrawing the sheath from the vein while the catheter is disposed within the sheath and peeling the sheath apart from the handle to the end portion of the sheath.

15. A method for venous access, comprising:
obtaining an introducer tool including a housing including a guidewire actuating mechanism configured to extend and retract a guidewire fixed to the guidewire actuating mechanism;
a peelable sheath including a handle, wherein the sheath is detachably coupled to the housing in an initial configuration of the introducer tool, and wherein the sheath is configured to peel apart from the handle to an end portion of the sheath; and
a needle fixed to the housing, wherein an end portion of the needle includes a beveled section and a dilator section;
puncturing a vein of a patient with the beveled section of the needle of the introducer tool to gain venous access by way of a puncture, wherein the end portion of the needle extends past the end portion of the sheath of the introducer tool for the puncturing of the vein;
dilating the puncture with the dilator section of the needle;
extending the guidewire into the vein past the end portion of the needle with the guidewire actuating mechanism;
introducing the sheath into the vein by sliding the sheath over the guidewire. activating a needle-safety mechanism of the introducer tool;
simultaneously withdrawing the needle from the puncture and enclosing at least the beveled section of the needle in a needle-safety housing of the needle-safety mechanism;
withdrawing the guidewire while leaving the sheath in place;
inserting a catheter into the vein through the sheath; and
removing the sheath from the vein, wherein removing the sheath includes withdrawing the sheath from the vein while the catheter is disposed within the sheath and peeling the sheath apart from the handle to the end portion of the sheath.

16. The introducer tool of claim 15, wherein the sheath has a pair of longitudinal seams or weakened areas enabling the sheath to peel apart from the handle to the end portion of the sheath.

17. The introducer tool of claim 15, wherein the sheath includes a polymer in which polymer chains of the polymer are longitudinally oriented along the sheath enabling the sheath to peel apart from the handle to the end portion of the sheath.
